(19) United States
(12) Patent Application Publication
(10) Pub. No.: US 2026/0338748 A1
(43) Pub. Date: Oct. 29, 2026

(54) METHOD FOR CALIBRATING VOLUME OF GAS MEASURING SYSTEM

(71) Applicant: Institute of Geology and Geophysics, Chinese Academy of Sciences, Beijing (CN)

(72) Inventors: Fei SU, Beijing (CN); Xuhang ZHANG, Beijing (CN); Huaiyu HE, Beijing (CN)

(21) Appl. No.: 19/466,515

(22) Filed: Feb. 2, 2026

(30) Foreign Application Priority Data
Feb. 13, 2025 (CN) 202510157591.1

(51) Int. Cl. G01N 33/00

(52) U.S. Cl. CPC G01N 33/0006; G01N 33/0014

(57) ABSTRACT

The present disclosure relates to the field of gas measurement and discloses a method for calibrating the volume of a gas measurement system, including the following steps: evacuating the system to remove residual gas and setting valves to an initial state; injecting high-purity He gas into the target area and causing the gas to fill the target pipeline segment through diffusion; utilizing a mass spectrometer to measure the He gas signal intensity of the pipeline segment closest to the mass spectrometer, and calculating the volume of the pipeline segment in combination with the known volume of the mass spectrometer; sequentially opening valves segment by segment in order from closest to farthest from the mass spectrometer, and recursively calculating the volume of each segment based on differences in signal intensity; finally recording and outputting the volume data of each pipeline segment and the total system volume data.

### TECHNICAL FIELD

The disclosure relates to the technical field of gas measurement, and in particular to a method for calibrating the volume of a gas measurement system.

### BACKGROUND

Gas measurement systems are widely used in scientific research and industrial applications, and the accurate calibration of the gas measurement system volume distribution is crucial for ensuring the precision of the system's measurement results. However, existing volume calibration methods are generally difficult to adapt to gas measurement systems with complex pipeline structures, such as multi-segment pipelines, irregular packing materials, and cold trap structures. In such systems, directly measuring the volume of each pipeline segment is often constrained by the pipeline shape, location, and the internal complex environment. These constraints lead to difficulties in accurately obtaining the actual volume of each pipeline segment.

In conventional methods, the system volume is typically calibrated through a one-time overall calibration or estimated based on the system's geometric structure. However, these methods exhibit significant limitations. Overall calibration fails to distinguish the specific distribution of volume across individual segments and obtains only the total system volume. However, geometric structure estimation is prone to significant deviations due to neglecting the internal complexity of pipelines or actual installation errors. Furthermore, because the measurement process is susceptible to gas mixture, signal interference, and equipment errors, existing methods also exhibit significant deficiencies in terms of precision and applicability.

Especially in scenarios with high requirements for volume parameters, such as rare gas measurement and high-precision gas flow control, existing techniques often fail to meet practical demands. Therefore, a technical method capable of adapting to complex pipeline systems, calibrating volume segment by segment, and ensuring accurate, reliable, and traceable calibration results is required to overcome the defects of existing techniques and provide support for the further optimization of gas measurement systems.

### SUMMARY

Aiming at the deficiencies of the existing techniques, the present disclosure provides a method for calibrating the volume of a gas measurement system. The method solves the problems of insufficient precision, poor adaptability in complex pipeline structures, and inability to obtain complete volume distribution data in existing gas measurement system volume calibration methods.

To achieve the above objectives, the present disclosure is implemented through the following technical solution: a method for calibrating the volume of a gas measurement system, includes the following steps:
S1, preparation work: evacuating the gas measurement system to remove residual gas inside the system, and setting all valves of the system to an initial state;
S2, calibrating the volume of the pipeline segment (V1) closest to the mass spectrometer:
S2.1, high-purity gas injection: injecting high-purity He gas into the mass spectrometer by diffusion; measuring the signal intensity of the He gas using the mass spectrometer; during the measurement process, evacuating all pipelines except the mass spectrometer to remove residual gas;
S2.2, back-diffusing the gas from the mass spectrometer into the target segment; measuring the signal intensity of the He gas again using the mass spectrometer, and calculating the volume of the pipeline segment in combination with the internal cavity volume of the mass spectrometer;
S2.3, repeating the above processes in S2.1 and S2.2 a plurality of times; obtaining the average value to determine the volume of the pipeline segment closest to the mass spectrometer;
S3, calibrating the volume of other pipeline segments:
S3.1, high-purity gas injection: injecting high-purity He gas into the entire pipeline;
S3.2, sequentially opening corresponding valves segment by segment in order from closest to farthest from the mass spectrometer, measuring the signal intensity difference via the mass spectrometer, and recursively calculating the volume of each segment;
S3.3, recording and outputting: recording the volume of each pipeline segment, and obtaining complete volume distribution data of the system; and
S3.4, repeating steps S3.1-S3.3 a plurality of times, and obtaining an average value.

Optionally, in the above steps, calibrating the volume of the pipeline segment closest to the mass spectrometer specifically includes:
by opening and closing corresponding valves, causing high-purity He gas to diffuse into the mass spectrometer;
measuring the signal intensity of He gas via the mass spectrometer, recording an initial signal value;
closing all other valves connected to the pipeline segment;
back-diffusing the gas from the mass spectrometer into the pipeline segment, measuring a back-diffusion signal value;
calculating the volume of the pipeline segment using the following formula:

$${v}_{1} = \frac{S 1 - S 2}{S 2} \times {v}_{M S}$$

where v₁ is the volume of the pipeline segment closest to the mass spectrometer to be calibrated, S1 is the signal intensity of He gas measured by the mass spectrometer, S2 is the signal intensity of He gas after back-diffusion from the mass spectrometer into the pipeline segment (V1) closest to the mass spectrometer, and v_{MS} is the internal cavity volume of the mass spectrometer.

Optionally, when calibrating the volume of other pipeline segments, proceeding segment by segment in order from closest to farthest from the mass spectrometer; when calibrating a certain pipeline segment, first diffusing the gas from the pipeline segment into the pipeline segment closest to the mass spectrometer (V1), and introducing the gas from the pipeline segment closest to the mass spectrometer (V1) into the mass spectrometer for measurement; calculating according to the following recursive formula:

$${v}_{i} = \frac{S i {g}_{i}}{S i {g}_{1} - S i {g}_{i}} \times {\sum }_{j = 1}^{i - 1} {v}_{j}$$

where vᵢ is the volume of the i-th pipeline segment to be calibrated, Sigᵢ is the signal intensity of He gas related to the i-th pipeline segment measured by the mass spectrometer, Sig₁ is the signal intensity of He gas in the initial calibration pipeline segment measured by the mass spectrometer, and

$${\sum }_{j = 1}^{i - 1} {v}_{j}$$

is the total volume of the first i−1 pipeline segments.

Optionally, in the calibration process, the injection amount of high-purity He gas is controlled within the range of 1×10⁻³ccSTP−5×10³ccSTP.

Optionally, the number of repeated measurements during volume calibration for each pipeline segment is at least 3, and the average value is taken as the final volume value for the segment.

Optionally, the gas measurement system includes a plurality of valves, pipelines, and a mass spectrometer, where

the valve GVTP and the valve GVIP1 are connected to a vacuum pump; the valves GV2-GV8 are sequentially connected to each pipeline segment of the gas measurement system for isolating or connecting each pipeline segment; the valve GVMS is connected to the inlet of the mass spectrometer, and the initial state of the system is as follows: GVTP, GVIP1, GV2, GV3, GV4, GV5, GV6, and GV7 are in an open state, and the remaining valves are in a closed state.

The present disclosure provides a method for calibrating the volume of a gas measurement system. The method has the following beneficial effects:
First, in the present disclosure, by adopting a segment-by-segment recursive calibration method combined with signal intensity measurement of the mass spectrometer and known reference volume parameters, the problem of result deviation caused by system complexity or accumulation of measurement errors in conventional overall calibration methods is avoided, thereby significantly improving the precision of volume calibration.
Second, in the present disclosure, through the technical solution of segment-by-segment isolation calibration, the method can flexibly adapt to gas measurement systems with complex pipeline structures, including special structures such as multi-segment pipelines, irregular packing materials, and cold traps, thereby ensuring the adaptability and accuracy of the calibration process and having a wide range of application.
Third, in the present disclosure, by recording, organizing, and outputting the volume of each pipeline segment and the total system volume, the method achieves traceability of volume distribution data, providing reliable foundational data support for subsequent experimental design, equipment calibration, and improvement of measurement precision.
Fourth, in the present disclosure, by precisely controlling the gas diffusion path and valve states to ensure independent connection between the target pipeline segment and the mass spectrometer, the possibility of gas mixture or interference from uncalibrated areas is reduced, thereby ensuring the reliability of signal measurement and the accuracy of volume calculation.
Fifth, in the present disclosure, by generating complete system volume distribution data, the method provides a scientific basis for the calibration and optimization of gas measurement systems. It is particularly suitable for application scenarios requiring high-precision volume parameter support, such as gas flow measurement and rare gas analysis, thereby further enhancing the practicality and functionality of the system.

### BRIEF DESCRIPTION OF THE DRAWINGS

FIG. 1 is a schematic flowchart of the method according to the present disclosure.

FIG. 2 is a schematic diagram of the gas measurement system according to the present disclosure.

### DETAILED DESCRIPTION OF THE EMBODIMENTS

The technical solutions in the embodiments of the present disclosure will be clearly and completely described below in conjunction with the accompanying drawings of the specification of the present disclosure. Obviously, the described embodiments are only a part of the embodiments of the present disclosure, and not all of them. All other embodiments obtained by a person of ordinary skill in the art based on the embodiments of the present disclosure without creative efforts shall fall within the scope of protection of the present disclosure.

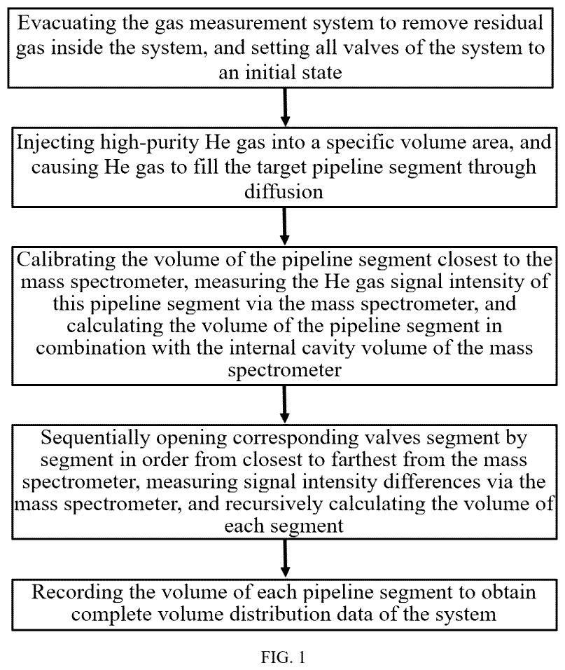

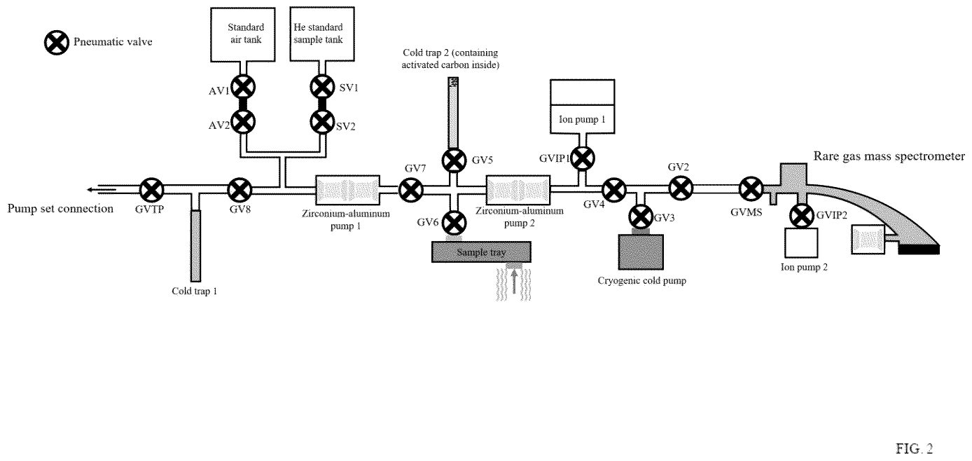

As shown in FIG. 1 and FIG. 2, the present disclosure provides a method for calibrating the volume of a gas measurement system. Through a segment-by-segment calibration approach, the method precisely measures the internal volume of each segment of the gas measurement system. It is applicable to complex gas measurement systems comprising multiple pipeline segments, valves, mass spectrometers, and other related components. This method can effectively address the problem of large errors in conventional volume calibration methods caused by the complexity and irregularity of the system's internal volume.

As shown in FIG. 1, a method for calibrating the volume of a gas measurement system includes the following steps:
S1, preparation work: evacuating the gas measurement system to remove residual gas inside the system, and setting all valves of the system to an initial state;
S2, calibrating the volume of the pipeline segment closest to the mass spectrometer:
S2.1, high-purity gas injection: injecting high-purity He gas into the mass spectrometer by diffusion; measuring the signal intensity of the He gas using the mass spectrometer; during the measurement process, evacuating all pipelines except the mass spectrometer to remove residual gas;
S2.2, back-diffusing the gas from the mass spectrometer into the target segment; measuring the signal intensity of the He gas again using the mass spectrometer, and calculating the volume of the pipeline segment in combination with the internal cavity volume of the mass spectrometer;
S2.3, repeating the above processes in S2.1 and S2.2 a plurality of times; obtaining the average value to determine the volume of the pipeline segment closest to the mass spectrometer;
S3, calibrating the volume of other pipeline segments:
S3.1, high-purity gas injection: injecting high-purity He gas into the entire pipeline;
S3.2, sequentially opening corresponding valves segment by segment in order from closest to farthest from the mass spectrometer, measuring the signal intensity difference via the mass spectrometer, and recursively calculating the volume of each segment;
S3.3, recording and outputting: recording the volume of each pipeline segment, and obtaining complete volume distribution data of the system; and
S3.4, repeating steps S3.1-S3.3 a plurality of times, and obtaining an average value.

The various steps of the method of the present disclosure are described in detail below.

For the step S1, in this embodiment, the step S1 primarily involves the preparation work of the gas measurement system to ensure the accuracy and stability of the subsequent calibration process.

Specifically, in this embodiment, a vacuum pumping operation is first performed on the gas measurement system to remove any residual gas and impurities inside the system, ensuring the cleanliness of the measurement environment. The vacuum pumping operation can be achieved by connecting a vacuum pump. The vacuum pump should have sufficient pumping speed and ultimate vacuum to meet the experimental requirements.

As an option, during specific implementation, a combination system of a mechanical pump and a molecular pump may be used to evacuate the pipeline. The mechanical pump is used for initially removing gas at atmospheric pressure, and the molecular pump is used for further reducing the gas pressure within the system to achieve a high vacuum state. It should be noted that the vacuum pumping operation should continue until the system reaches a preset vacuum condition. For example, the system vacuum can reach <10⁻³ Pa to ensure that the influence of residual gas on the subsequent high-purity He gas calibration process is minimized.

In a possible implementation, the connection between the vacuum pump and the system is controlled by a main valve (such as GVTP). After opening the main valve, the vacuum pump begins evacuating the system. Once the required vacuum level is achieved, the main valve is closed to isolate the vacuum pump from the system. This operational approach can reduce interference from external gas leakage on the system vacuum.

It should be noted that during the vacuum pumping process, in order to avoid gas residue caused by irregular packing materials (such as adsorbents in a cold trap) or complex pipeline structures within the system, a heating operation may be employed to assist the vacuum pumping. For example, by applying appropriate heating to the pipeline surface to raise the system temperature to 100 °C-200 °C, the presence of residual gas or adsorbed gas can be further reduced.

In this embodiment, each valve within the system needs to be set to a specific initial state according to the requirements of the experimental design. The state settings of these valves are crucial for the diffusion path of He gas and the signal acquisition of the mass spectrometer. Specifically, in the initial state, all valves directly related to the target pipeline segments (for example, GVTP, GVIP1) should be in an open state, while valves related to non-target areas or non-operational modules (for example, GVMS or valves connected to other pipeline segments) should be in a closed state. It should be understood that this valve state configuration ensures controllable gas flow paths during subsequent operations and avoids interference from non-calibration areas.

In an exemplary operation, the system can be divided into several independent sub-areas. Each sub-area is isolated or connected by valves to ensure that vacuum pumping acts only on the target area. Other areas can be independently evacuated by opening their respective valves when needed. This segmented vacuum pumping method is particularly suitable for systems containing complex pipeline structures.

Understandably, the initial state settings of the aforementioned valves are adjustable. Depending on the actual structure of different systems and the experimental requirements, the specific open or closed states of the valves can be appropriately adjusted. It should be ensured that the target areas are in an operational state and non-target areas are in an isolated state.

As an optimization measure, the system vacuum can be monitored in real-time using pressure sensors after setting the valve states. Exemplarily, pressure sensors may be arranged at the connection between the target area and the vacuum pump, or at the inlet of the mass spectrometer, to ensure that the vacuum conditions of each target area meet the experimental requirements. If a pressure sensor detects that the pressure value does not meet the preset requirement, correction can be made by extending the vacuum pumping time or inspecting potential leak points.

It should be noted that the aforementioned preparation work plays a foundational role in the technical solution of the present disclosure. Its core purpose is to ensure the internal environment of the system is in a pure state, providing reliable assurance for the subsequent He gas injection and calibration process.

For the step S2 in this embodiment, the step S2 primarily involves the injection operation of high-purity He gas to ensure the target pipeline segment of the gas measurement system is filled with high-purity calibration gas, thereby laying the foundation for subsequent volume calibration.

Specifically, in this embodiment, the injection of high-purity He gas is completed through a preset gas injection path. The gas source can be a high-purity He gas cylinder or a gas generation device. As an option, the high-purity He gas used should meet the purity requirement; the purity usually needs to reach ≥99.999% in order to avoid interference from other impurity gases with the mass spectrometry measurement results.

In a possible implementation, high-purity He gas is introduced into the system through a connected injection pipeline (for example, the area between SV1 and SV2). It should be noted that the injection path for He gas should be as short as possible to reduce gas diffusion delay and gas loss caused by an excessively long path. During specific operation, the SV1 valve is opened to allow high-purity He gas to be injected into the predetermined injection volume at a stable flow rate. After being maintained for a certain period (such as 2 min), the SV1 valve is closed. Understandably, this gas injection method, through control of time and flow rate, ensures that the He gas within the injection volume can be fully filled.

It should be noted that after He gas injection, the gas gradually fills the target pipeline segment through diffusion. In this embodiment, the diffusion operation is achieved by opening valves associated with the target pipeline segment. For example, by opening the SV2 valve and other control valves (such as GV2) connected to the target pipeline segment, the injected He gas diffuses from the area between SV1 and SV2 into the target pipeline segment. The duration of the diffusion operation is generally controlled within 1-2 min to ensure He gas can fully enter the target area. As an option, the pressure change in the target pipe section can be monitored by a pressure sensor, and when the pressure reaches equilibrium, the diffusion process is complete.

In one possible embodiment, to further improve diffusion efficiency, a heating operation may be performed on the target pipeline segment. For example, a heating tape or heating jacket may be disposed externally to the pipeline segment to raise the segment temperature to a range of 30 °C-50 °C, thereby enhancing the diffusion rate and uniformity of the He gas. It should be noted that this heating operation is particularly applicable to pipeline segments containing complex packing materials, such as irregular structures like cold traps.

The control of the diffusion path is a key technical point in this embodiment. Specifically, to ensure accuracy of the He gas diffusion range, other valves not related to the target pipeline segment (for example, GV3 and subsequent valves) should be in a closed state during the diffusion process. This operational approach can effectively isolate non-target pipeline segments, ensuring diffusion occurs only within the target pipeline segment, thereby avoiding interference from gases in other areas.

Understandably, during the diffusion process, if the diffusion range of He gas cannot be fully controlled, it may lead to gas leakage into non-target areas or uncalibrated areas, thereby affecting subsequent measurement accuracy. Therefore, to further reduce the complexity of the diffusion path, in some embodiments, the system may be divided into multiple independent modules to gradually guide gas diffusion into the target pipeline segment. Exemplarily, pressure within the target pipeline segment may be used to determine whether diffusion is complete based on the pressure difference with its adjacent pipeline segments. For example, if the pressure within the target pipeline segment reaches a stable value close to the pressure of the injection area, it may be considered that the diffusion process is complete.

It should be noted that the completion marker of the diffusion process is the He gas within the target pipeline segment reaching a stable state. The gas state at this time is crucial for subsequent mass spectrometry measurement. In this case, to avoid the effect of He gas concentration fluctuations on the measurement, a waiting period (for example, 1-2 min) may be applied after the diffusion is completed to ensure completely uniform gas distribution within the target pipeline segment.

As a possible improvement, after the diffusion is completed, a preliminary measurement of the He gas concentration within the target pipeline segment may be performed using the mass spectrometer to ensure the gas purity and concentration meet the expected requirements. If the measurement result deviates from the set value, high-purity He gas may be reinjected and the above diffusion steps repeated until the gas within the target pipeline segment reaches a stable state.

In the step S2 of this embodiment, through precise control of He gas injection and diffusion, the purity and uniformity of the gas within the target pipeline segment can be ensured, providing reliable experimental conditions for subsequent volume calibration.

For the step S2, in this embodiment, the step S2 involves the calibration operation of the volume of the pipeline segment closest to the mass spectrometer. By measuring the signal intensity of He gas and combining it with the known volume parameters of the mass spectrometer, the actual volume of the target pipeline segment is calculated, providing a basis for subsequent calibration of other pipeline segments.

Specifically, when calibrating the volume of the target pipeline segment closest to the mass spectrometer, the gas path of the system first needs to be adjusted to ensure only this target pipeline segment is connected to the mass spectrometer, while valves of other pipeline segments are in a closed state. It should be noted that at this time, the target pipeline segment is filled with uniformly diffused high-purity He gas, and other parts of the system have been evacuated to a vacuum state to avoid interference from gases in other areas on the measurement signal.

In a possible implementation, by opening the control valve (for example, GVMS) between the target pipeline segment (such as V1) and the mass spectrometer and closing valves of other pipeline segments (for example, GV2, GVIP2, etc.), He gas is introduced from the target pipeline segment into the mass spectrometer. In an exemplary operation, the mass spectrometer detects the signal intensity of He gas through its internal sensor and records this signal value, noted as the first measurement signal value S₁.

As an option, the operating time of the mass spectrometer may be set according to the specific circumstances of the system structure, generally controlled to measure 10 cycles to ensure signal detection stability. After completion of the first signal measurement, the He gas within the mass spectrometer is back-diffused into the target pipeline segment. The back-diffusion operation is implemented by closing the valve corresponding to the target pipeline segment (such as GV2) and opening the connection valve between the mass spectrometer and the target pipeline segment (such as GVMS), allowing the He gas within the mass spectrometer to flow back into the target pipeline segment. Understandably, the back-diffusion operation can effectively eliminate the influence of residual gas within the mass spectrometer on subsequent measurements.

In a possible implementation, after the above back-diffusion operation, signal measurement using the mass spectrometer is performed again, and the signal value after back-diffusion is recorded, noted as the second measurement signal value S2. It should be noted that the stability of the second signal measurement is crucial for the accuracy of volume calculation. Therefore, after completion of the back-diffusion, a wait of 30 s-1 min is typically required to ensure uniform gas distribution.

In this embodiment, the volume of the target pipeline segment is calculated using the following formula:

$${v}_{1} = \frac{S 1 - S 2}{S 2} \times {v}_{M S}$$

where
v₁ is the volume of the pipeline segment closest to the mass spectrometer;
S1 is the signal intensity of He gas from the first measurement;
S2 is the signal intensity of He gas from the second measurement after back-diffusion;
v_{MS} is the internal cavity volume of the mass spectrometer, a known parameter provided by the mass spectrometer manufacturer.

As an optimization method, when calculating the volume, multiple repeated measurements (for example, 3 times or more) may be performed on the values of the first and second signal measurements, and then the average value is taken to reduce the impact of experimental errors on the calculation results. Understandably, repeated measurements help eliminate deviations caused by equipment fluctuations or gas flow variations, thereby improving the accuracy of volume calibration.

In exemplary operations, the internal cavity volume of the mass spectrometer is typically a fixed value, such as 1000 cc, but the actual volume may vary depending on different models. It should be noted that the internal cavity volume of the mass spectrometer serves as a known reference value, and its accuracy is crucial for calculating the volume of the target pipeline segment. Therefore, the mass spectrometer should be calibrated before use to ensure the provided reference value is accurate.

As a possible extension, in order to verify the reliability of the volume calculation result, the calculated volume v₁ of the target pipeline segment may be compared with a theoretical estimated value. The theoretical estimated value can be calculated based on the geometric dimensions (such as length and inner diameter) of the target pipeline segment. If the difference between the two is significant, the mass spectrometry signal measurement process should be re-examined for potential abnormalities, such as gas leakage or signal detection errors.

It should be noted that the volume calibration of the target pipeline segment closest to the mass spectrometer serves as the foundation for the subsequent calibration of other pipeline segments, and its result directly affects the accuracy of subsequent recursive calculations. Therefore, during the operation, the rigor of the calibration process and the reliability of the data should be ensured. In the step S2, through signal intensity measurement of high-purity He gas combined with known reference volume, precise calibration of the target pipeline segment volume is achieved, providing a reliable basis for volume calibration of the entire gas measurement system.

For the step S3, in this embodiment, the step S3 mainly involves the operation of calibrating the volume of other pipeline segments farther from the mass spectrometer. Through a segment-by-segment recursive approach, the volume of each pipeline segment is sequentially calculated. In this step, using the volume of the pipeline segment closest to the mass spectrometer as a known basis and combining it with the relationship of changes in mass spectrometry signal intensity, the calibration of the volumes of the remaining pipeline segments is completed.

Specifically, in this embodiment, when calibrating the volumes of pipeline segments farther from the mass spectrometer (for example, V2, V3, etc.), measurement of the target pipeline segments needs to be performed segment by segment in the order from closest to farthest from the mass spectrometer. Understandably, this segment-by-segment recursive operational approach can avoid interference from system complexity on the calibration process and gradually improve calibration accuracy.

In a possible implementation, the valve states of the system first need to be adjusted to ensure the current target pipeline segment is connected to the mass spectrometer and the already calibrated pipeline segments, while other pipeline segments that have not yet been calibrated remain isolated. For example, when calibrating the second pipeline segment V2, the valve connected to V2 (such as GV2) needs to be opened, while valves related to other pipeline segments (such as V3 and farther segments) (such as GV3, GV4, etc.) need to be closed. Through this valve operation, He gas is ensured to diffuse only from the current target pipeline segment into the already calibrated part and finally enter the mass spectrometer.

Exemplarily, when the target pipeline segment (such as V3) is to be calibrated, valve GV3 needs to be opened to allow gas from the target pipeline segment to diffuse into the already calibrated area (V1, V2). Subsequently, valve GV2 is closed, requiring only the gas within the pipeline segment closest to the mass spectrometer to enter the mass spectrometer for measurement. The obtained signal value is recorded as the signal value Sigᵢ for the current segment. As an option, the signal value may be repeatedly measured (for example, measured 3 times repeatedly) and the average value taken to reduce deviations caused by environmental fluctuations or equipment errors. When calibrating the volume of each target pipeline segment, the gas inside it needs to be diffused into the pipeline segment closest to the mass spectrometer, and only the gas within the pipeline segment closest to the mass spectrometer is taken for measurement. This approach can reduce error propagation during data calculation and improve the accuracy of volume calibration.

It should be noted that during the calibration process of each pipeline segment, the already calibrated portion within the diffusion path should always be maintained in a vacuum state. This can be achieved by opening the vacuum pump control valve (for example, GVTP) to evacuate the already calibrated portion. For example, when calibrating V2, the already calibrated portion of pipeline segment V1 needs to be evacuated to ensure no residual gas remains, thereby avoiding interference from gas mixture on the measurement of the current segment.

In this embodiment, the volume of the target pipeline segment is calculated using the following formula:

$${v}_{i} = \frac{{Sig}_{i}}{{Sig}_{1} - {Sig}_{i}} \times {\sum }_{j = 1}^{i - 1} {v}_{j}$$

where
vᵢ is the volume of the i-th pipeline segment to be calibrated;
Sigᵢ is the signal intensity of He gas within the i-th pipeline segment measured by the mass spectrometer;
Sig₁ is the signal intensity of He gas in the initial calibration pipeline segment (such as V1) measured by the mass spectrometer;

$${\sum }_{j = 1}^{i - 1} {v}_{j}$$

is the total volume of the first i−1 pipeline segments.
Specifically, when calibrating V2, the above formula is utilized:

$${v}_{2} = \frac{{Sig}_{i}}{{Sig}_{1} - {Sig}_{2}} \times {v}_{1}$$

where v₁ is the volume of the already calibrated first pipeline segment, and Sig₂ is the signal intensity of the current segment.

In a possible optimized implementation, calibration accuracy may be improved by adjusting the diffusion time. For example, during the diffusion process, by opening GV2 and maintaining for 2 min, He gas fully diffuses into the connected area between V2 and V1. After the diffusion is completed, GV2 is closed to allow gas within V1 to enter the mass spectrometer for measurement, ensuring the diffusion range matches the measurement path.

It should be noted that when calibrating the third segment (such as V3) and farther pipeline segments, recursive calculation should be performed based on the volume of the already calibrated portion (such as V1 and V2).

Understandably, this recursive calculation approach ensures the calibration result of each segment's volume is based on the measurement data of preceding segments, avoiding potential cumulative errors from independent calibration.

As an improvement measure, the target pipeline segment may be measured multiple times, and the measurement results may be subjected to fitting analysis, thereby further enhancing the precision of the calculation results. For example, errors caused by signal fluctuations or changes in experimental conditions can be reduced by performing linear fitting on measurement results from different diffusion times.

In exemplary operations, for pipeline segments containing multiple complex structures (such as cold traps or packing materials), diffusion efficiency can also be improved through heating operations, thereby ensuring the stability of signal intensity. The heating temperature can be controlled within 30 °C-50 °C, with the specific temperature adjustable according to the system material and structural characteristics.

It should be noted that the process of calibrating the volumes of other pipeline segments should proceed segment by segment. The calibration of each segment is based on the already calibrated volumes, strictly following the sequence from nearest to farthest. This sequential calibration method not only improves overall measurement accuracy, but also allows verification of the calibration results for the current segment through the progressively accumulated volume values.

This embodiment achieves precise calibration of the volumes of other pipeline segments farther from the mass spectrometer through recursive calculation of mass spectrometry signal intensity, providing reliable data support for the complete volume distribution of the entire gas measurement system.

For the step S3.3, in this embodiment, the step S3.3 mainly involves recording and outputting the volume calibration results of the gas measurement system. By organizing and summarizing the calibration data of each pipeline segment's volume, a complete system volume distribution is obtained, thereby providing an accurate reference basis for subsequent gas measurement, analysis, and equipment calibration.

Specifically, in this embodiment, after completing the calibration of all pipeline segment volumes, the volume values v₁, v₂, . . . , vₙ of each pipeline segment are recorded according to their connection sequence relative to the mass spectrometer, and the system total volume Vₜₒₜₐₗ is obtained through cumulative calculation. The calculation formula is as follows:

$${V}_{total} = {\sum }_{i = 1}^{n} {v}_{i}$$

where
vᵢ is the volume of the i-th pipeline segment;
n is the total number of pipeline segments in the system.

As an option, when recording volume data, a tabular format may be used to separately record the number, connection position, volume value, and relevant conditions during measurement (such as ambient temperature, gas type, diffusion time, etc.) of each pipeline segment. This information can provide a traceability basis for subsequent analysis.

It should be noted that the recording of system volume distribution can be selected in different output forms according to specific requirements. For example, in a laboratory environment, volume data is usually saved in the form of electronic spreadsheets or databases to facilitate subsequent consultation and use; in an industrial environment, volume data can also be directly output to a display screen or printing device through an integrated control system of the equipment.

In a possible implementation, the recorded data can be automatically organized and processed through software programs. Exemplarily, measurement data can be transmitted to data processing software through communication interfaces (such as RS232, USB, etc.) between sensors and computers, and volume calculation, data storage, and output operations are completed within the software. For example, the software can automatically calculate the volume of each segment based on the input signal intensity values and calibration formulas, and generate a system volume distribution diagram. This automated processing approach can improve the efficiency of data recording and output, avoiding errors possibly arising from manual recording.

Understandably, during data output, uncertainty factors in the calibration process should be clearly annotated. For example, calibration accuracy information can be appended to the output results, such as the measurement error range of signal intensity or the uncertainty in volume calculation for different segments. Specifically, the error range can be calculated based on the signal intensity measurement values in the calibration formula and the sensitivity of the measurement equipment. For example, for the error range ΔSigᵢ of the measurement value Sigᵢ, the calculation error range Δvᵢ for volume can be estimated through the error propagation formula.

It should be noted that for complex pipeline systems, spatial distribution information can also be appended to the calibration results. For example, a three-dimensional modeling tool can be used to combine the volume values of each pipeline segment with their spatial positions, generating a visual volume distribution model. This visualization result can help technicians more intuitively understand the volume distribution characteristics of the system and provide a basis for the design of subsequent gas measurement experiments.

In some embodiments, the output of calibration results can also be integrated with the control system of the mass spectrometer itself to achieve linkage with subsequent measurement processes. For example, calibration results are directly stored in the built-in memory of the mass spectrometer, making the calibration results reference data for subsequent gas measurements. During operation, the mass spectrometer can automatically perform signal calibration based on the stored volume distribution data, thereby improving the accuracy of measurement results.

In this embodiment, the recording, organizing, and outputting of the volume values of each pipeline segment is taken as the final step of the calibration method. This not only achieves the acquisition of complete system volume distribution data but also provides reliable foundational support for subsequent applications.

In summary, the present disclosure evacuates the gas measurement system, injects high-purity gas, utilizes a mass spectrometer to measure gas signal intensity segment by segment, combines with the system structure and calculation formulas to sequentially calibrate the volume of each pipeline segment, and ultimately obtains complete system volume distribution data. This method adopts a recursive calculation approach, calibrating sequentially from the pipeline segment closest to the mass spectrometer toward the distal segments, thereby avoiding error accumulation issues associated with direct volume measurement of complex systems. The method exhibits characteristics of high precision and high reliability, provides technical support for the calibration and optimization of gas measurement systems, and is applicable to complex pipeline volume calibration requirements in laboratory and industrial fields.

To better understand the present disclosure, the above method is described in detail below in conjunction with a specific embodiment.

### EMBODIMENT

This embodiment uses the system in FIG. 2 as an example to describe the calibration method of the present disclosure in detail. In FIG. 2, the volume between GVMS and GV2 is defined as V1; the volume among GV2, GV3, and GV4 is defined as V2; the volume of the cryogenic cold pump is defined as V3; the volume among GV4, GV5, GV6, GV7, and GVIP1 is defined as V4; the volume of cold trap 2 is defined as V5; the volume of the sample tray is defined as V6; the volume among GV7, GV8, AV2, and SV2 is defined as V7; the volume between GVTP and GV8 is defined as V8.

Prior to commencing calibration, the states of the system valves are as follows: GVTP, GVIP1, GV2, GV3, GV4, GV5, GV6, and GV7 are in the open state, while all other valves are in the closed state.
1. The volume v₁ of the system V1 between GV2 and GVMS is calibrated by means of back-diffusion.
a) Introducing high-purity He gas into the volume between SV1 and SV2. The specific operation is: opening SV1, closing SV1 after 2 min.
b) Diffusing the He standard gas into the entire system. The specific operation is: closing GVTP and GVIP1, opening GV8 and SV2, closing SV2 after 2 min. At this point, the gas has diffused from the area between SV1 and SV2 into the entire pipeline.
c) Performing mass spectrometry measurement. Closing GVIP2, opening GVMS, closing GVMS after 1 min, mass spectrometer measuring ⁴He, recording the test result as S1. Simultaneously, opening GVTP to evacuate residual gas within the system pipeline, maintaining it in a vacuum state.
d) After completion of the mass spectrometry measurement, back-diffusing the gas from within the mass spectrometer into the pipeline segment closest to the mass spectrometer (V1), i.e., the portion from GV2 to GVMS. The specific operation is: closing GV2, opening GVMS. After 1 min, the mass spectrometer measures ⁴He, recording the test result as S2. After the measurement is completed, closing GVMS, opening GV2 and GVIP2. The internal cavity volume v_{MS} of the mass spectrometer is provided by the mass spectrometer manufacturer, thus

$${v}_{1} = \frac{S 1 - S 2}{S 2} * {v}_{MS} .$$

e) Repeating the above steps three or more times, taking the average of multiple v₁ values as the definitive value for the volume v₁ of V1 between GV2 and GVMS.
2. The volumes v₂-v₈ of the respective parts V2-V8 are calibrated.
a) Introducing high-purity He gas into the volume between SV1 and SV2: opening SV1, closing SV1 after 2 min.
b) Diffusing the He standard gas into the entire system. The specific operation is: closing GVTP and GVIP1, opening GV8 and SV2, closing SV2 after 2 min. At this point, the gas has diffused from the area between SV1 and SV2 into the entire pipeline. Subsequently, closing all valves (GV1, GV2, GV3, GV4, GV5, GV6, GV7). In this way, the gas is isolated and stored separately within V1-V8.
c) Closing GVIP2, opening GVMS, allowing gas from the V1 portion, i.e., between GV2 and GVMS, to enter the mass spectrometer. Closing GVMS after 1 min, the mass spectrometer measures ⁴He, recording the test result as Sig1. After the measurement ends, opening GVIP2 and GVMS to evacuate residual gas, closing GVMS after 5 min.
d) Calibrating the volume v₂. The specific operation is: opening GV2, closing GV2 after 2 min. Closing GVIP2, opening GVMS, closing GVMS after 1 min, the mass spectrometer measures ⁴He, recording the test result as Sig2. After the measurement ends, opening GVIP2, GVMS, and GV2, closing GVMS after 5 min. The formula is

$${v}_{2} = \frac{Sig 2}{Sig 1 - Sig 2} \times {v}_{1} .$$

e) Calibrating the volume v₃. The specific operation is: opening GV3, closing GV2 after 2 min. Closing GVIP2, opening GVMS, closing GVMS after 1 min, the mass spectrometer measures ⁴He, recording the test result as Sig3. After the measurement ends, opening GVIP2, GVMS, and GV2, closing GVMS after 5 min. The formula is

$${v}_{3} = \frac{Sig 3}{Sig 1 - Sig 3} \times ( {v}_{1} + {v}_{2} ) .$$

f) Calibrating the volume v₄. The specific operation is: opening GV4, closing GV2 after 2 min. Opening GVIP1, closing GVIP2, opening GVMS, closing GVMS after 1 min, opening GV2, the mass spectrometer measures ⁴He, recording the test result as Sig4. After the measurement ends, opening GVIP2. The formula is

$${v}_{4} = \frac{Sig 4}{Sig 1 - Sig 4} \times ( {v}_{1} + {v}_{2} + {v}_{3} ) .$$

g) Calibrating the volume v₅. The specific operation is: Closing GVIP1, opening GV5, closing GV2 after 2 min, opening GVIP1. Closing GVIP2, opening GVMS, closing GVMS after 1 min, opening GV2, the mass spectrometer measures ⁴He, recording the test result as Sig5. After the measurement ends, opening GVIP2. The formula is

$${v}_{5} = \frac{Sig 5}{Sig 1 - Sig 5} \times ( {v}_{1} + {v}_{2} + {v}_{3} + {v}_{4} ) .$$

h) Calibrating the volume v₆. The specific operation is: closing GVIP1, opening GV6, closing GV2 after 2 min, opening GVIP1. Closing GVIP2, opening GVMS, closing GVMS after 1 min, opening GV2, the mass spectrometer measures ⁴He, recording the test result as Sig6. After the measurement ends, opening GVIP2. The formula is

$${v}_{6} = \frac{Sig 6}{Sig 1 - Sig 6} \times ( {v}_{1} + {v}_{2} + {v}_{3} + {v}_{4} + {v}_{5} ) .$$

i) Calibrating the volume v₇. The specific operation is: closing GVIP1, opening GV7, closing GV2 after 2 min, opening GVIP1. Closing GVIP2, opening GVMS, closing GVMS after 1 min, opening GV2, the mass spectrometer measures ⁴He, recording the test result as Sig7. After the measurement ends, opening GVIP2. The formula is

$${v}_{7} = \frac{Sig 7}{Sig 1 - Sig 7} \times ( {v}_{1} + {v}_{2} + {v}_{3} + {v}_{4} + {v}_{5} + {v}_{6} ) .$$

j) Calibrating the volume v₈. The specific operation is: closing GVIP1, opening GV8, closing GV2 after 2 min, opening GVIP1. Closing GVIP2, opening GVMS, closing GVMS after 1 min, opening GV2, the mass spectrometer measures ⁴He, recording the measured signal as Sig8. After the measurement ends, opening GVIP2. Closing GV8, opening GVIP1. The formula is

$${v}_{8} = \frac{Sig 8}{Sig 1 - Sig 8} \times ( {v}_{1} + {v}_{2} + {v}_{3} + {v}_{4} + {v}_{5} + {v}_{6} + {v}_{7} ) .$$

k) Repeating the above steps more than three times, taking the average value as the definitive value for the volume of each part.

While embodiments of the present disclosure have been shown and described, it will be understood by those of ordinary skill in the art that various changes, modifications, substitutions, and alterations can be made to these embodiments without departing from the principles and spirit of the present disclosure. The scope of the present disclosure is defined by the appended claims and their equivalents.

1. A method for calibrating a volume of a gas measurement system, comprising the following steps:
S1, evacuating the gas measurement system to remove residual gas, and resetting the valves to an initial state;
S2, calibrating the volume of the pipeline segment closest to the mass spectrometer:
S2.1, diffusing and injecting high-purity He gas into the mass spectrometer; measuring the signal intensity of He gas using the mass spectrometer; during measurement, evacuating all pipelines except the mass spectrometer to remove residual gas;
S2.2, back-diffusing the gas from within the mass spectrometer to the target segment; performing a second measurement of He gas signal intensity; and calculating the volume of the pipeline segment in combination with the internal cavity volume of the mass spectrometer;
S2.3, repeating S2.1 and S2.2 a plurality of times, and obtaining the average value to obtain the volume of the pipeline segment closest to the mass spectrometer;
S3, calibrating the volume of other pipeline segments:
S3.1, injecting high-purity He gas into the entire pipeline;
S3.2, sequentially opening corresponding valves segment by segment in order from closest to farthest from the mass spectrometer, measuring the signal intensity difference via the mass spectrometer, and recursively calculating the volume of each segment;
S3.3, recording the volume of each pipeline segment, and obtaining complete volume distribution data of the system;
S3.4, repeating steps S3.1-S3.3 a plurality of times, and obtaining an average value;
calibrating the volume of the pipeline segment closest to the mass spectrometer includes:
opening and closing corresponding valves to cause high-purity He gas to diffuse into the mass spectrometer;
measuring the He gas signal intensity and recording an initial signal value;
closing all other valves connected to the pipeline segment;
back-diffusing the gas from within the mass spectrometer to the pipeline segment and measuring the signal value;
calculating the volume of the pipeline segment:
$${v}_{1} = \frac{S 1 - S 2}{S 2} \times {v}_{MS}$$
wherein v₁ is the volume of the pipeline segment closest to the mass spectrometer to be calibrated, S1 is the signal intensity of He gas measured by the mass spectrometer, S2 is the signal intensity of He gas after back-diffusion from the mass spectrometer into the pipeline segment closest to the mass spectrometer, and v_{MS} is the internal cavity volume of the mass spectrometer;
when calibrating the volume of other pipeline segments, proceeding segment by segment in order from closest to farthest from the mass spectrometer; when calibrating a certain pipeline segment, first diffusing the gas from the pipeline segment into the pipeline segment closest to the mass spectrometer, and introducing the gas from the pipeline segment closest to the mass spectrometer into the mass spectrometer for measurement; calculating according to the recursive formula:
$${v}_{i} = \frac{{Sig}_{i}}{{Sig}_{1} - {Sig}_{i}} \times {\sum }_{j = 1}^{i - 1} {v}_{j}$$
wherein vᵢ is the volume of the i-th pipeline segment to be calibrated, Sigᵢ is the signal intensity of He gas related to the i-th pipeline segment measured by the mass spectrometer, Sig₁ is the signal intensity of He gas in the initial calibration pipeline segment measured by the mass spectrometer, and
$${\sum }_{j = 1}^{i - 1} {v}_{j}$$
is the total volume of the first i−1 pipeline segments;
during the calibration process of each pipeline segment, the calibrated pipeline segments within the diffusion path are maintained in a vacuum state.

2. The method for calibrating the volume of the gas measurement system according to claim 1, wherein in the calibration process, the injection amount of high-purity He gas is controlled within the range of 1×10⁻³ccSTP−5×10⁻³ ccSTP.

3. The method for calibrating the volume of the gas measurement system according to claim 1, wherein the number of repeated measurements during volume calibration for each pipeline segment is at least 3, and the average value is taken as the final volume value for the segment.

4. The method for calibrating the volume of the gas measurement system according to claim 1, wherein the gas measuring system comprises a plurality of valves, pipelines and a mass spectrometer, wherein
the valve GVTP and the valve GVIP1 are connected to a vacuum pump; the valves GV2-GV8 are sequentially connected to each pipeline segment of the gas measurement system for isolating or connecting each pipeline segment; the valve GVMS is connected to the inlet of the mass spectrometer, and the initial state of the system is as follows: GVTP, GVIP1, GV2, GV3, GV4, GV5, GV6, and GV7 are in an open state, and the remaining valves are in a closed state.
